(19) United States
(12) Patent Application Publication
(10) Pub. No.: US 2025/0380740 A1
(43) Pub. Date: Dec. 18, 2025

(54) SMOKING SUBSTITUTE SYSTEM

(71) Applicant: Imperial Tobacco Limited, Bristol (GB)

(72) Inventors: Kate Ferrie, Liverpool (GB); Ross Shenton, Liverpool (GB); Chris Lord, Liverpool (GB)

(21) Appl. No.: 19/185,953

(22) Filed: Apr. 22, 2025

Related U.S. Application Data
(63) Continuation of application No. 17/481,889, filed on Sep. 22, 2021, now Pat. No. 12,329,201.
Application No. 17/481,889 is a continuation of application No. PCT/EP2020/056769, filed on Mar. 13, 2020.
Application No. PCT/EP2020/056769 is a continuation of application No. PCT/EP2020/056772, filed on Mar. 13, 2020.
Application No. PCT/EP2020/056772 is a continuation of application No. PCT/EP2020/056776, filed on Mar. 13, 2020.
Application No. PCT/EP2020/056776 is a continuation of application No. PCT/EP2020/056777, filed on Mar. 13, 2020.
Application No. PCT/EP2020/056777 is a continuation of application No. PCT/EP2020/056782, filed on Mar. 13, 2020.
Application No. PCT/EP2020/056782 is a continuation-in-part of application No. PCT/EP2020/056784, filed on Mar. 13, 2020.
Application No. PCT/EP2020/056784 is a continuation of application No. PCT/EP2020/056786, filed on Mar. 13, 2020.
Application No. PCT/EP2020/056786 is a continuation of application No. PCT/EP2020/056788, filed on Mar. 13, 2020.
Application No. PCT/EP2020/056788 is a continuation of application No. PCT/EP2020/056792, filed on Mar. 13, 2020.
Application No. PCT/EP2020/056792 is a continuation of application No. PCT/EP2020/056818, filed on Mar. 13, 2020.
Application No. PCT/EP2020/056818 is a continuation of application No. PCT/EP2020/056822, filed on Mar. 13, 2020.
Application No. PCT/EP2020/056822 is a continuation of application No. PCT/EP2020/056823, filed on Mar. 13, 2020.
Application No. PCT/EP2020/056823 is a continuation of application No. PCT/EP2020/056825, filed on Mar. 13, 2020.
Application No. PCT/EP2020/056825 is a continuation of application No. PCT/EP2020/056836, filed on Mar. 13, 2020.
Application No. PCT/EP2020/056836 is a continuation of application No. PCT/EP2020/056837, filed on Mar. 13, 2020.
Application No. PCT/EP2020/056837 is a continuation of application No. PCT/EP2020/056838, filed on Mar. 13, 2020.
Application No. PCT/EP2020/056838 is a continuation of application No. PCT/EP2020/056854, filed on Mar. 13, 2020.
Application No. PCT/EP2020/056854 is a continuation of application No. PCT/EP2020/056861, filed on Mar. 13, 2020.
Application No. PCT/EP2020/056861 is a continuation of application No. PCT/EP2020/056863, filed on Mar. 13, 2020.
Application No. PCT/EP2020/056863 is a continuation of application No. PCT/EP2020/056868, filed on Mar. 13, 2020.
Application No. PCT/EP2020/056868 is a continuation of application No. PCT/EP2020/056870, filed on Mar. 13, 2020.

(30) Foreign Application Priority Data
Mar. 22, 2019 (EP) 19020137.6
Mar. 22, 2019 (EP) 19020138.4
Mar. 22, 2019 (EP) 19020142.6
Mar. 22, 2019 (EP) 19020147.5
Mar. 22, 2019 (EP) 19020150.9
Mar. 22, 2019 (EP) 19020153.3
Mar. 22, 2019 (EP) 19020155.8
Mar. 22, 2019 (EP) 19020156.6
Mar. 22, 2019 (EP) 19020158.2
Mar. 22, 2019 (EP) 19020159.0
Mar. 22, 2019 (EP) 19020164.0
Mar. 22, 2019 (EP) 19020168.1
Mar. 22, 2019 (EP) 19020169.9
Mar. 22, 2019 (EP) 19020173.1
Mar. 22, 2019 (EP) 19020176.4
Mar. 22, 2019 (EP) 19020179.8
Mar. 22, 2019 (EP) 19020183.0
Mar. 22, 2019 (EP) 19020185.5
Mar. 22, 2019 (EP) 19020189.7
Mar. 22, 2019 (EP) 19020197.0
Mar. 22, 2019 (EP) 19020201.0
Mar. 22, 2019 (EP) 19020203.6
Mar. 22, 2019 (EP) 19020206.9
Mar. 22, 2019 (EP) 19020209.3
Mar. 22, 2019 (EP) 19020210.1
Mar. 22, 2019 (EP) 19020212.7
Mar. 22, 2019 (EP) 19020213.5
Mar. 22, 2019 (EP) 19020216.8
Mar. 22, 2019 (EP) 19020223.4
Feb. 14, 2020 (EP) 20157500.8

(51) Int. Cl. A24F 40/46; A24F 40/20; A24F 40/70; A46B 15/00; A61M 11/04; B25B 27/14; H05B 3/06

(52) U.S. Cl. CPC A24F 40/46; A24F 40/20; A24F 40/70; A46B 15/0055; B25B 27/14; H05B 3/06; A46B 2200/3073; A61M 11/042; A61M 2209/10

(57) ABSTRACT

A smoking substitute device is disclosed. The device comprises a main body (209), a heater (223) and a cap (210) engagable with the main body and movable between a closed position in which it substantially encloses the heater and an open position wherein at least a portion of the heater is exposed. The device further comprises a sensor (229) for detecting a position of the cap, and a controller configured to control the device in response to the detection the position of the cap.

### CROSS REFERENCE TO RELATED APPLICATIONS/INCORPORATION BY REFERENCE STATEMENT

This application is a continuation of 17/481,889 filed on Sep. 22, 2021. This application is a non-provisional application claiming benefit to the international application no. PCT/EP2020/056769 filed on Mar. 13, 2020, which claims priority to EP 19020153.3 filed on Mar. 22, 2019 and to EP 20157500.8 filed on Feb. 14, 2020. This application also claims benefit to the international application no. PCT/EP2020/056772 filed on Mar. 13, 2020, which claims priority to EP 19020150.9 filed on Mar. 22, 2019. This application also claims benefit to the international application no. PCT/EP2020/056776 filed on Mar. 13, 2020, which claims priority to EP 19020137.6 filed on Mar. 22, 2019, EP 19020138.4 filed on Mar. 22, 2019, EP 19020159.0 filed on Mar. 22, 2019, EP 19020173.1 filed on Mar. 22, 2019, EP 19020176.4 filed on Mar. 22, 2019, EP 19020185.5 filed on Mar. 22, 2019, EP 19020189.7 filed on Mar. 22, 2019, EP 19020210.1 filed on Mar. 22, 2019, EP 19020213.5 filed on Mar. 22, 2019, and EP 19020169.9 filed on Mar. 22, 2019. This application also claims benefit to the international application no. PCT/EP2020/056777 filed on Mar. 13, 2020, which claims priority to EP 19020183.0 filed on Mar. 22, 2019. This application also claims benefit to the international application no. PCT/EP2020/056782 filed on Mar. 13, 2020, which claims priority to EP 19020179.8 filed on Mar. 22, 2019. This application also claims benefit to the international application no. PCT/EP2020/056784 filed on Mar. 13, 2020, which claims priority to EP 19020216.8 filed on Mar. 22, 2019. This application also claims benefit to the international application no. PCT/EP2020/056786 filed on Mar. 13, 2020, which claims priority to EP 19020212.7 filed on Mar. 22, 2019. This application also claims benefit to the international application no. PCT/EP2020/056788 filed on Mar. 13, 2020, which claims priority to EP 19020209.3 filed on Mar. 22, 2019. This application also claims benefit to the international application no. PCT/EP2020/056792 filed on Mar. 13, 2020, which claims priority to EP 19020203.6 filed on Mar. 22, 2019. This application also claims benefit to the international application no. PCT/EP2020/056818 filed on Mar. 13, 2020, which claims priority to EP 19020168.1 filed on Mar. 22, 2019. This application also claims benefit to the international application no. PCT/EP2020/056822 filed on Mar. 13, 2020, which claims priority to EP 19020155.8 filed on Mar. 22, 2019. This application also claims benefit to the international application no. PCT/EP2020/056823 filed on Mar. 13, 2020, which claims priority to EP 19020156.6 filed on Mar. 22, 2019. This application also claims benefit to the international application no. PCT/EP2020/056825 filed on Mar. 13, 2020, which claims priority to EP 19020159.0 filed on Mar. 22, 2019. This application also claims benefit to the international application no. PCT/EP2020/056836 filed on Mar. 13, 2020, which claims priority to EP 19020164.0 filed on Mar. 22, 2019. This application also claims benefit to the international application no. PCT/EP2020/056837 filed on Mar. 13, 2020, which claims priority to EP 19020223.4 filed on Mar. 22, 2019. This application also claims benefit to the international application no. PCT/EP2020/056838 filed on Mar. 13, 2020, which claims priority to EP 19020158.2 filed on Mar. 22, 2019. This application also claims benefit to the international application no. PCT/EP2020/056854 filed on Mar. 13, 2020, which claims priority to EP 19020147.5 filed on Mar. 22, 2019. This application also claims benefit to the international application no. PCT/EP2020/056861 filed on Mar. 13, 2020, which claims priority to EP 19020197.0 filed on Mar. 22, 2019. This application also claims benefit to the international application no. PCT/EP2020/056863 filed on Mar. 13, 2020, which claims priority to EP 19020142.6 filed on Mar. 22, 2019. This application also claims benefit to the international application no. PCT/EP2020/056868 filed on Mar. 13, 2020, which claims priority to EP 19020201.0 filed on Mar. 22, 2019. This application also claims benefit to the international application no. PCT/EP2020/056870 filed on Mar. 13, 2020, which claims priority to EP 19020206.9 filed on Mar. 22, 2019. The entire contents of each of the above referenced applications are hereby incorporated herein by reference in their entirety.

### FIELD OF THE DISCLOSURE

The present invention relates to a smoking substitute system and particularly, although not exclusively, to a smoking substitute system comprising a smoking substitute device having a cap.

### BACKGROUND

The smoking of tobacco is generally considered to expose a smoker to potentially harmful substances. It is generally thought that a significant amount of the potentially harmful substances is generated through the heat caused by the burning and/or combustion of the tobacco and the constituents of the burnt tobacco in the tobacco smoke itself.

Conventional combustible smoking articles, such as cigarettes, typically comprise a cylindrical rod of tobacco comprising shreds of tobacco which is surrounded by a wrapper, and usually also a cylindrical filter axially aligned in an abutting relationship with the wrapped tobacco rod. The filter typically comprises a filtration material which is circumscribed by a plug wrap. The wrapped tobacco rod and the filter are joined together by a wrapped band of tipping paper that circumscribes the entire length of the filter and an adjacent portion of the wrapped tobacco rod. A conventional cigarette of this type is used by lighting the end opposite to the filter, and burning the tobacco rod. The smoker receives mainstream smoke into their mouth by drawing on the mouth end or filter end of the cigarette.

Combustion of organic material such as tobacco is known to produce tar and other potentially harmful by-products. There have been proposed various smoking substitute systems (or “substitute smoking systems”) in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or flavorings without, or with fewer of, the odor and health risks associated with traditional smoking.

In general, smoking substitute systems are intended to provide a substitute for the rituals of smoking, whilst providing the user with a similar experience and satisfaction to those experienced with traditional smoking and with combustible tobacco products. Some smoking substitute systems use smoking substitute articles (also referred to as a “consumable”) that are designed to resemble atraditional cigarette and are cylindrical in form with a mouthpiece at one end.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories.

There are a number of different categories of smoking substitute systems, each utilizing a different smoking substitute approach.

One approach for a smoking substitute system is the so-called Heated Tobacco (“HT”) approach in which tobacco (rather than an “e-liquid”) is heated or warmed to release vapor. HT is also known as “heat not burn” (“HNB”). The tobacco may be leaf tobacco or reconstituted tobacco. The vapor may contain nicotine and/or flavorings. In the HT approach the intention is that the tobacco is heated but not burned, i.e., the tobacco does not undergo combustion.

A typical HT smoking substitute system may include a device and a consumable. The consumable may include the tobacco material. The device and consumable may be configured to be physically coupled together. In use, heat may be imparted to the tobacco material by a heating element of the device, wherein airflow through the tobacco material causes components in the tobacco material to be released as vapor. A vapor may also be formed from a carrier in the tobacco material (this carrier may for example include propylene glycol and/or vegetable glycerin) and additionally volatile compounds released from the tobacco. The released vapor may be entrained in the airflow drawn through the tobacco.

As the vapor passes through the consumable (entrained in the airflow) from the location of vaporization to an outlet of the consumable (e.g., a mouthpiece), the vapor cools and condenses to form an aerosol for inhalation by the user. The aerosol will normally contain the volatile compounds.

In HT smoking substitute systems, heating as opposed to burning the tobacco material is believed to cause fewer, or smaller quantities, of the more harmful compounds ordinarily produced during smoking. Consequently, the HT approach may reduce the odor and/or health risks that can arise through the burning, combustion and pyrolytic degradation of tobacco.

Whilst the heating element is not heated to a temperature that burns the tobacco, when heated, its temperature does present a safety hazard to users. That is, if a user were to come into direct contact with the heater it could cause significant injury to the user.

Thus, there may be a need for improved design of smoking substitute systems, in particular HT smoking substitute systems, to enhance the user experience by inclusion of safety features, and improve the function of the HT smoking substitute system.

The present disclosure has been devised in the light of the above considerations.

### SUMMARY OF THE DISCLOSURE

At its most general, the present invention relates to a smoking substitute device having a heating element that is deactivated when a cap of the device is moved to expose the heater.

According to a first aspect of the present invention, there is provided a smoking substitute device a main body; a heater; a cap engageable with the main body and movable between a closed position in which it substantially encloses the heater and an open position wherein at least a portion of the heater is exposed; a sensor for detecting a position of the cap; and a controller configured to control the device in response to the detection of the position of the cap.

Providing a device having a sensor for detecting the position of a cap and a controller able to control the device in response that detection allows the device to function differently when, for example, the cap is removed. This may allow the implementation of safety controls when the cap is removed (which exposes the heater).

The term “substantially encloses” does not require that the cap fully encloses the heater. An opening may remain for inserting e.g., a smoking substitute article into the device (for engagement) with the heater. However, such an opening (due to its size) would generally not allow a user to touch the heater (so as to present a safety hazard). In this respect, one would not consider the heater to be exposed.

Optional features will now be set out. These are applicable singly or in any combination with any aspect.

The controller may be configured to prevent activation of the heater when the sensor detects that the cap is in the open position. The control of the device may alternatively comprise reducing the temperature of the heater (but not fully deactivating the heater). The control of the device may alternatively comprise locking a user input of the device to prevent user interaction with the device. The controller may be configured not to respond to user input (e.g., by activating the heater) when the cap is detected as being in the open position. The detection of the cap being in the open position may be in the form of the sensor not detecting the cap in the closed position. That is, the sensor may only be configured to detect whether the cap is in the closed position, such that an absence of that detection is a detection of the cap being in the open position.

In the open position, the cap may be (e.g., fully) disengaged from the main body. Alternatively, the cap may be engaged with the main body of the device in the open and closed positions (and may remain engaged with the body between those positions). The cap may be slidably or rotatably engaged with the device so as to be movable between the closed and open positions. In the open position, an opening (exposing the heater) may be formed between the cap and the main body.

The device may comprise a power source for supplying power to the heater. The controller may prevent activation of the heater by preventing a supply of power from the power source to the heater. For example, the controller may prevent the supply of power in circumstances where supply of power would normally be provided (e.g., such as an activation input from a user via a button, switch, touchscreen, etc.).

The device may comprise a magnet disposed on the cap or main body. The device may comprise a sensor disposed on the other of the cap or the main body (i.e., the magnet and sensor may be disposed on different components). The sensor may be configured to detect the presence of the magnet when the cap is engaged with the main body.

The magnet may be disposed on the cap and the sensor may be disposed on the main body. The body may comprise a cavity for receipt of at least a portion of the cap. The sensor may be mounted to or at a wall defining the cavity. The magnet may be mounted to the at least a portion of the cap received in the cavity. Thus, the magnet and sensor may be adjacent or in proximity when the at least a portion of the cap is received in the cavity.

The sensor may be a Hall effect sensor. In other embodiments the sensor may be e.g., a light sensor. For example, the light sensor may receive light through an opening in the device, and that light may be blocked when the cap is in the closed position. Alternatively, the sensor may comprise a switch that is activated when the cap is moved into or out of the closed position.

The device may comprise a magnet disposed on the cap or main body and a ferromagnetic element (e.g., a metal element such as a plate or block) disposed on the other of the cap or the main body. The ferromagnetic element and magnet may be arranged so as to magnetically interact when the cap is in the closed position. That is, the ferromagnetic element and the magnet may align when the cap is in the closed position so as to be adjacent to or proximate one another. The magnet may be configured such that the magnetic interaction retains the cap on the body. The ferromagnetic element may form part of the housing of the cap. The ferromagnetic element and the magnet may interact so as to align the cap with respect to the body. The magnet may be the same magnet (as discussed above) that is detected by the sensor when the cap is in the closed position. One of the ferromagnetic element and magnet may be disposed on a wall defining a cavity of the body, and the other may be disposed on a portion of the cap received in the cavity.

The magnets and/or sensor may be received in respective mounting recesses of their respective components. The magnets and/or sensor may be attached to their respective components (or walls of their respective components) by way of an adhesive.

An end of the main body may be configured for engagement with an aerosol-forming article (i.e., the end of the main body comprising the heater). The main body may be configured for engagement with a heated tobacco (HT) consumable (or heat-not-burn (HNB) consumable) The terms “heated tobacco” and “heat-not-burn” are used interchangeably herein to describe a consumable that is of the type that is heated rather than combusted (or are used interchangeably to describe a device for use with such a consumable). The cavity (which received the at least a portion of the cap) may also be configured for receipt of at least a portion of the consumable (i.e., for engagement with the consumable). The aerosol-forming article may be of the type that comprises an aerosol former (e.g., carried by an aerosol-forming substrate).

The heater may be for heating the aerosol-forming article. The heater may comprise a heating element, which may be in the form of a rod that extends from the main body of the device. The heating element may extend from the end of the main body that is configured for engagement with the aerosol-forming article.

The heater (and thus the heating element) may be rigidly mounted to the main body. The heating element may be elongate so as to define a longitudinal axis and may, for example, have a transverse profile (i.e., transverse to a longitudinal axis of the heating element) that is substantially circular (i.e., the heating element may be generally cylindrical). Alternatively, the heating element may have a transverse profile that is rectangular (i.e., the heater may be a “blade heater”). The heating element may alternatively be in the shape of a tube (i.e., the heater may be a “tube heater”). The heating element may take other forms (e.g., the heating element may have an elliptical transverse profile). The shape and/or size (e.g., diameter) of the transverse profile of the heating element may be generally consistent for the entire length (or substantially the entire length) of the heating element. When the cap is in the open position (but remains engaged with the main body) the heating element may be exposed laterally (e.g., intermediate the ends of the heating element).

The heating element may be between 15 mm and 25 mm long, e.g., between 18 mm and 20 mm long, e.g., around 19 mm long. The heating element may have a diameter of between 1.5 mm and 2.5 mm, e.g., a diameter between 2 mm and 2.3 mm, e.g., a diameter of around 2.15 mm.

The heating element may be formed of ceramic. The heating element may comprise a core (e.g., a ceramic core) comprising Al2O3. The core of the heating element may have a diameter of 1.8 mm to 2.1 mm, e.g., between 1.9 mm and 2 mm. The heating element may comprise an outer layer (e.g., an outer ceramic layer) comprising Al2O3. The thickness of the outer layer may be between 160 μm and 220 μm, e.g., between 170 μm and 190 μm, e.g., around 180 μm. The heating element may comprise a heating track, which may extend longitudinally along the heating element. The heating track may be sandwiched between the outer layer and the core of the heating element. The heating track may comprise tungsten and/or rhenium. The heating track may have a thickness of around 20 μm.

As mentioned above, the heating element may be located in the cavity (of the device), and may extend (e.g., along a longitudinal axis) from an internal base of the cavity towards an opening of the cavity. The length of the heating element (i.e., along the longitudinal axis of the heater) may be less than the depth of the cavity. Hence, the heating element may extend for only a portion of the length of the cavity. That is, the heating element may not extend through (or beyond) the opening of the cavity.

The heating element may be configured for insertion into an aerosol-forming article (e.g., a HT consumable) when an aerosol-forming article is received in the cavity. In that respect, a distal end (i.e., distal from a base of the heating element where it is mounted to the device) of the heating element may comprise a tapered portion, which may facilitate insertion of the heating element into the aerosol-forming article. The heating element may fully penetrate an aerosol-forming article when the aerosol-forming article is received in the cavity. That is, the entire length, or substantially the entire length, of the heating element may be received in the aerosol-forming article.

The heating element may have a length that is less than, or substantially the same as, an axial length of an aerosol-forming substrate forming part of an aerosol-forming article (e.g., a HT consumable). Thus, when such an aerosol-forming article is engaged with the device, the heating element may only penetrate the aerosol-forming substrate, rather than other components of the aerosol-forming article. The heating element may penetrate the aerosol-forming substrate for substantially the entire axial length of the aerosol forming-substrate of the aerosol-forming article. Thus, heat may be transferred from (e.g., an outer circumferential surface of) the heating element to the surrounding aerosol-forming substrate, when penetrated by the heating element. That is, heat may be transferred radially outwardly (in the case of a cylindrical heating element) or e.g., radially inwardly (in the case of a tube heater).

Where the heater is a tube heater, the heating element of the tube heater may surround at least a portion of the cavity. When the portion of the aerosol-forming article is received in the cavity, the heating element may surround a portion of the aerosol-forming article (i.e., so as to heat that portion of the aerosol-forming article). In particular, the heating element may surround an aerosol forming substrate of the aerosol-forming article. That is, when an aerosol-forming article is engaged with the device, the aerosol forming substrate of the aerosol-forming article may be located adjacent an inner surface of the (tubular) heating element. When the heating element is activated, heat may be transferred radially inwardly from the inner surface of the heating element to heat the aerosol forming substrate.

The cavity may comprise a (e.g., circumferential) wall (or walls) and the (tubular) heating element may extend around at least a portion of the wall(s). In this way, the wall may be located between the inner surface of the heating element and an outer surface of the aerosol-forming article. The wall (or walls) of the cavity may be formed from a thermally conductive material (e.g., a metal) to allow heat conduction from the heating element to the aerosol-forming article. Thus, heat may be conducted from the heating element, through the cavity wall (or walls), to the aerosol-forming substrate of an aerosol-forming article received in the cavity.

The cap may define at least a portion of the cavity of the device. That is, the cavity may be fully defined by the cap, or each of the cap and main body may define a portion of the cavity. Where the cap fully defines the cavity, the cap may comprise an aperture for receipt of the heating element into the cavity (when the cap is in the closed position). The cap may comprise an opening to the cavity. The opening may be configured for receipt of at least a portion of an aerosol-forming article. That is, an aerosol-forming article may be inserted through the opening and into the cavity (so as to be engaged with the device).

The cap may be configured such that when an aerosol-forming article is engaged with the device (e.g., received in the cavity), only a portion of the aerosol-forming article is received in the cavity. That is, a portion of the aerosol-forming article (not received in the cavity) may protrude from (i.e., extend beyond) the opening. This (protruding) portion of the aerosol-forming article may be a terminal (e.g., mouth) end of the aerosol-forming article, which may be received in a user's mouth for the purpose of inhaling aerosol formed by the device.

As above, the device may comprise a power source. The device may alternatively be connectable to a power source (e.g., a power source separate to the device). As set forth above, the power source may be electrically connectable to the heater. In that respect, altering (e.g., toggling) the electrical connection of the power source to the heater may affect a state of the heater. For example, toggling the electrical connection of the power source to the heater may toggle the heater between an on state and an off state. The power source may be a power store. For example, the power source may be a battery or rechargeable battery (e.g., a lithium-ion battery).

The device may comprise an input connection (e.g., a USB port, Micro USB port, USB-C port, etc.). The input connection may be configured for connection to an external source of electrical power, such as a mains electrical supply outlet. The input connection may, in some cases, be used as a substitute for an internal power source (e.g., battery or rechargeable battery). That is, the input connection may be electrically connectable to the heater (for providing power to the heater). Hence, in some forms, the input connection may form at least part of the power source of the device.

Where the power source comprises a rechargeable power source (such as a rechargeable battery), the input connection may be used to charge and recharge the power source.

The device may comprise a user interface (UI). In some embodiments the UI may include input means to receive operative commands from the user. The input means of the UI may allow the user to control at least one aspect of the operation of the device. In some embodiments the input means may comprise a power button to switch the device between an on state and an off state. As above, the controller may render an input ineffective when the cap is detected as being in the open position.

In some embodiments the UI may additionally or alternatively comprise output means to convey information to the user. In some embodiments the output means may comprise a light to indicate a condition of the device (and/or the aerosol-forming article) to the user. The condition of the device (and/or aerosol-forming article) indicated to the user may comprise a condition indicative of the operation of the heater. For example, the condition may comprise whether the heater is in an off state or an on state. The condition may also be the position of the cap (e.g., the closed and/or open position). For example, the UI may indicate to a user that the cap is in an open position. This may only be indicated to a user when the user attempts to activate the heater. In some embodiments, the UI unit may comprise at least one of a button, a display, a touchscreen, a switch, a light, and the like. For example, the output means may comprise one or more (e.g., two, three, four, etc.) light-emitting diodes (“LEDs”) that may be located on the main body of the device.

The device may further comprise a puff sensor (e.g., airflow sensor), which form part of the input means of the UI. The puff sensor may be configured to detect a user drawing on an end (i.e., a terminal (mouth) end) of the aerosol-forming article. The puff sensor may, for example, be a pressure sensor or a microphone. The puff sensor may be configured to produce a signal indicative of a puff state. The signal may be indicative of the user drawing (an aerosol from the aerosol-forming article) such that it is e.g., in the form of a binary signal. Alternatively, or additionally, the signal may be indicative of a characteristic of the draw (e.g., a flow rate of the draw, length of time of the draw, etc.).

The controller may comprise a microcontroller that may e.g., be mounted on a printed circuit board (PCB). The controller may also comprise a memory, e.g., non-volatile memory. The memory may include instructions, which, when implemented, may cause the controller to perform certain tasks or steps of a method. Where the device comprises an input connection, the controller may be connected to the input connection.

As set forth above, the controller may be configured to control the operation of the heater (and e.g., the heating element). Thus, the controller may be configured to control vaporization of an aerosol forming part of an aerosol-forming article engaged with the device. The controller may be configured to control the voltage applied by power source to the heater. For example, the controller may be configured to toggle between applying a full output voltage (of the power source) to the heater and applying no voltage to the heater. Alternatively, or additionally, the control unit may implement a more complex heater control protocol.

The device may further comprise a voltage regulator to regulate the output voltage supplied by the power source to form a regulated voltage. The regulated voltage may subsequently be applied to the heater.

In some embodiments, where the device comprises a UI, the controller may be operatively connected to one or more components of the UI. The controller may be configured to receive command signals from an input means of the UI. The controller may be configured to control the heater in response to the command signals. For example, the controller may be configured to receive “on” and “off” command signals from the UI and, in response, may control the heater so as to be in a corresponding on or off state. As above, when the cap is in the open position, the controller may not respond to such “on” and “off” command signals in the usual manner.

The controller may be configured to send output signals to a component of the UI. The UI may be configured to convey information to a user, via an output means, in response to such output signals (received from the controller). For example, where the device comprises one or more LEDs, the LEDs may be operatively connected to the controller. Hence, the controller may be configured to control the illumination of the LEDs (e.g., in response to an output signal). For example, the controller may be configured to control the illumination of the LEDs according to (e.g., an on or off) state of the heater.

Where the device comprises a sensor in addition to the cap sensor (e.g., a puff/airflow sensor), the controller may be operatively connected to this further sensor. The controller may be configured to receive a signal from the sensor (e.g., indicative of a condition of the device and/or engaged aerosol-forming article). The controller may be configured to control the heater, or an aspect of the output means, based on the signal from the further sensor.

The device may comprise a wireless interface configured to communicate wirelessly (e.g., via Bluetooth (e.g., a Bluetooth low-energy connection) or Wi-Fi) with an external device. Similarly, the input connection may be configured for wired connection to an external device so as to provide communication between the device and the external device.

The external device may be a mobile device. For example, the external device may be a smart phone, tablet, smart watch, or smart car. An application (e.g., app) may be installed on the external device (e.g., mobile device). The application may facilitate communication between the device and the external device via the wired or wireless connection.

The wireless or wired interface may be configured to transfer signals between the external device and the controller of the device. In this respect, the controller may control an aspect of the device in response to a signal received from an external device. Alternatively, or additionally, an external device may respond to a signal received from the device (e.g., from the controller of the device).

In a second aspect there is provided a method of controlling a smoking substitute device, the method comprising detecting a state of a cap of the device and controlling a heater of the device in response to the detected state of the cap.

The state of the cap may comprise whether the cap is in an open or closed position. In the open position, the heater may be exposed. In the closed position the heater may be substantially enclosed by the cap. In the open position the cap may be (e.g., fully) disengaged from the device (e.g., a main body of the device).

Controlling the heater may comprise preventing activation of the heater when the cap is in the open position (or is disengaged from the device).

In a third aspect, there is provided a system (e.g., a smoking substitute system) comprising a device according to the first and an aerosol-forming article. The aerosol-forming article may comprise an aerosol-forming substrate at an upstream end of the aerosol-forming article.

Conveniently, the article may be in the form of a smoking substitute article, e.g., heated tobacco (HT) consumable (also known as a heat-not-burn (HNB) consumable).

As used herein, the terms “upstream” and “downstream” are intended to refer to the flow direction of the vapor/aerosol i.e., with the downstream end of the article/consumable being the mouth end or outlet where the aerosol exits the consumable for inhalation by the user. The upstream end of the article/consumable is the opposing end to the downstream end.

The aerosol-forming substrate is capable of being heated to release at least one volatile compound that can form an aerosol. The aerosol-forming substrate may be located at the upstream end of the article/consumable.

In order to generate an aerosol, the aerosol-forming substrate comprises at least one volatile compound that is intended to be vaporized/aerosolized and that may provide the user with a recreational and/or medicinal effect when inhaled. Suitable chemical and/or physiologically active volatile compounds include the group consisting of: nicotine, cocaine, caffeine, opiates and opioids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, Amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia califomica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

The plant material may be tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above-mentioned tobaccos.

The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon).

The aerosol-forming substrate may comprise a gathered sheet of homogenized (e.g., paper/slurry recon) tobacco or gathered shreds/strips formed from such a sheet.

The aerosol-forming substrate may comprise one or more additives selected from humectants, flavorants, fillers, aqueous/non-aqueous solvents and binders.

The flavorant may be provided in solid or liquid form. It may include menthol, licorice, chocolate, fruit flavor (including e.g., citrus, cherry etc.), vanilla, spice (e.g., ginger, cinnamon) and tobacco flavor. The flavorant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g., between 6 and 9 mm or 6 and 8 mm e.g., around 7 mm. It may have an axial length of between 10 and 15 mm e.g., between 11 and 14 mm such as around 12 or 13 mm.

The article/consumable may comprise at least one filter element. There may be a terminal filter element at the downstream/mouth end of the article/consumable.

The or at least one of the filter element(s) (e.g., the terminal filter element) may be comprised of cellulose acetate or polypropylene tow. The at least one filter element (e.g., the terminal filter element) may be comprised of activated charcoal. The at least one filter element (e.g., the terminal element) may be comprised of paper. The or each filter element may be at least partly (e.g., entirely) circumscribed with a plug wrap e.g., a paper plug wrap.

The terminal filter element (at the downstream end of the article/consumable) may be joined to the upstream elements forming the article/consumable by a circumscribing tipping layer e.g., a tipping paper layer. The tipping paper may have an axial length longer than the axial length of the terminal filter element such that the tipping paper completely circumscribes the terminal filter element plus the wrapping layer surrounding any adjacent upstream element.

In some embodiments, the article/consumable may comprise an aerosol-cooling element which is adapted to cool the aerosol generated from the aerosol-forming substrate (by heat exchange) before being inhaled by the user.

The article/consumable may comprise a spacer element that defines a space or cavity between the aerosol-forming substrate and the downstream end of the consumable. The spacer element may comprise a cardboard tube. The spacer element may be circumscribed by the (paper) wrapping layer.

According to a fourth aspect of the present invention, there is provided a method of using the system according to the third aspect, the method comprising inserting the aerosol-forming article into the device; and heating the article using the heater of the device.

In some embodiments the method may comprise inserting the article into a cavity within a body of the device and penetrating the article with the heating element of the device upon insertion of the article.

The invention includes the combination of the aspects and preferred features described except where such a combination is clearly impermissible or expressly avoided.

The skilled person will appreciate that except where mutually exclusive, a feature or parameter described in relation to any one of the above aspects may be applied to any other aspect. Furthermore, except where mutually exclusive, any feature or parameter described herein may be applied to any aspect and/or combined with any other feature or parameter described herein.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1A is a schematic of a smoking substitute system.

FIG. 1B is a schematic of a variation of the smoking substitute system of FIG. 1A.

FIG. 2A is a front view of a first embodiment of a smoking substitute system with the consumable engaged with the device.

FIG. 2B is a front view of the first embodiment of the smoking substitute system with the consumable disengaged from the device.

FIG. 2C is a section view of the consumable of the first embodiment of the smoking substitute system.

FIG. 2D is a detailed view of an end of the device of the first embodiment of the smoking substitute system.

FIG. 2E is a section view of the first embodiment of the substitute smoking system.

FIG. 2F is a section view of a portion of the main body of the first embodiment with the cap in an open position.

FIG. 2G is section view of a portion of the first embodiment with the cap in a closed position.

### DETAILED DESCRIPTION OF THE FIGURES

Aspects and embodiments of the present invention will now be discussed with reference to the accompanying figures. Further aspects and embodiments will be apparent to those skilled in the art. All documents mentioned in this text are incorporated herein by reference.

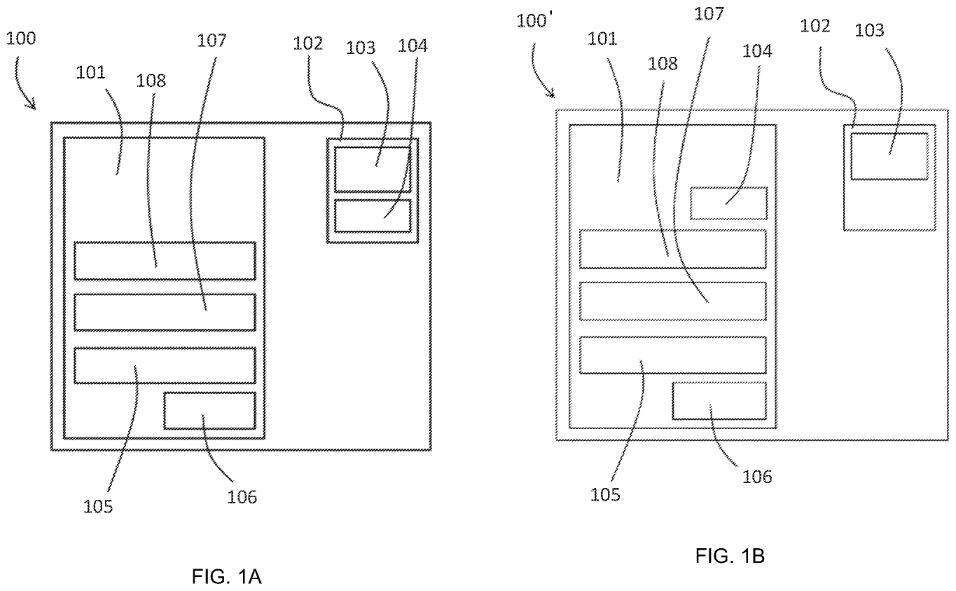

FIG. 1A is a schematic providing a general overview of a smoking substitute system 100. The system 100 includes a substitute smoking device 101 and an aerosol-forming article in the form of a consumable 102, which comprises an aerosol former 103. The system is configured to vaporize the aerosol former by heating the aerosol former 103 (so as to form a vapor/aerosol for inhalation by a user).

In the illustrated system, the heater 104 forms part of the consumable 102 and is configured to heat the aerosol former 103. In this variation, the heater 104 is electrically connectable to the power source 105, for example, when the consumable 102 is engaged with the device 101. Heat from the heater 104 vaporizes the aerosol former 103 to produce a vapor. The vapor subsequently condenses to form an aerosol, which is ultimately inhaled by the user.

The system 100 further comprises a power source 105 that forms part of the device 101. In other embodiments the power source 105 may be external to (but connectable to) the device 101. The power source 105 is electrically connectable to the heater 104 such that it is able to supply power to the heater 104 (i.e., for the purpose of heating the aerosol former 103). Thus, control of the electrical connection of the power source 105 to the heater 104 provides control of the state of the heater 104. The power source 105 may be a power store, for example a battery or rechargeable battery (e.g., a lithium-ion battery).

The system 100 further comprises an I/O module comprising a connector 106 (e.g., in the form of a USB port, Micro USB port, USB-C port, etc.). The connector 106 is configured for connection to an external source of electrical power, e.g., a mains electrical supply outlet. The connector 106 may be used in substitution for the power source 105. That is the connector 106 may be electrically connectable to the heater 104 so as to supply electricity to the heater 104. In such embodiments, the device may not include a power source, and the power source of the system may instead comprise the connector 106 and an external source of electrical power (to which the connector 106 provides electrical connection).

In some embodiments, the connector 106 may be used to charge and recharge the power source 105 where the power source 105 includes a rechargeable battery.

The system 100 also comprises a user interface (UI) 107. Although not shown, the UI 107 may include input means to receive commands from a user. The input means of the UI 107 allows the user to control at least one aspect of the operation of the system 100. The input means may, for example, be in the form of a button, touchscreen, switch, microphone, etc.

The UI 107 also comprises output means to convey information to the user. The output means may, for example, comprise lights (e.g., LEDs), a display screen, speaker, vibration generator, etc.

The system 100 further comprises a controller 108 that is configured to control at least one function of the device 101. In the illustrated embodiment, the controller 108 is a component of the device 101, but in other embodiments may be separate from (but connectable to) the device 101. The controller 108 is configured to control the operation of the heater 104 and, for example, may be configured to control the voltage applied from the power source 105 to the heater 104. The controller 108 may be configured to toggle the supply of power to the heater 104 between an on state, in which the full output voltage of the power source 105 is applied to the heater 104, and an off state, in which the no voltage is applied to the heater 104.

Although not shown, the system 100 may also comprise a voltage regulator to regulate the output voltage from the power source 105 to form a regulated voltage. The regulated voltage may then be applied to the heater 104.

In addition to being connected to the heater 104, the controller 108 is operatively connected to the UI 107. Thus, the controller 108 may receive an input signal from the input means of the UI 107.

Similarly, the controller 108 may transmit output signals to the UI 107. In response, the output means of the UI 107 may convey information, based on the output signals, to a user. The controller also comprises a memory 109, which is a non-volatile memory. The memory 109 includes instructions, which, when implemented, cause the controller to perform certain tasks or steps of a method.

FIG. 1B is a schematic showing a variation of the system 100 of FIG. 1A. In the system 100′ of FIG. 1B, the heater 104 forms part of the device 101, rather than the consumable 102. In this variation, the heater 104 is electrically connected to the power source 105.

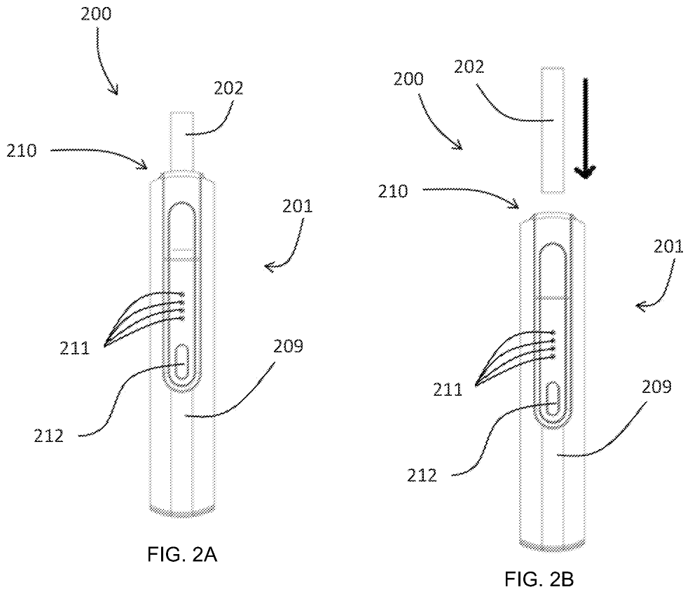

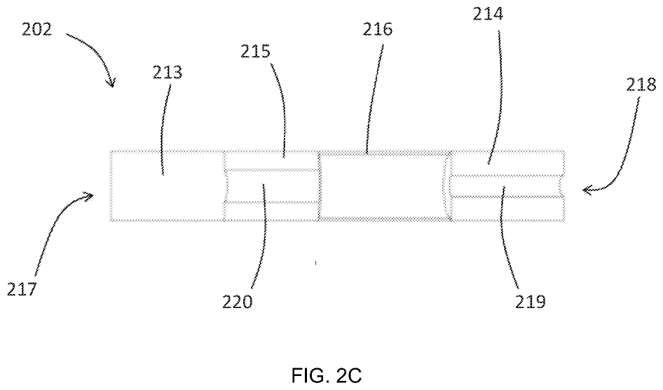

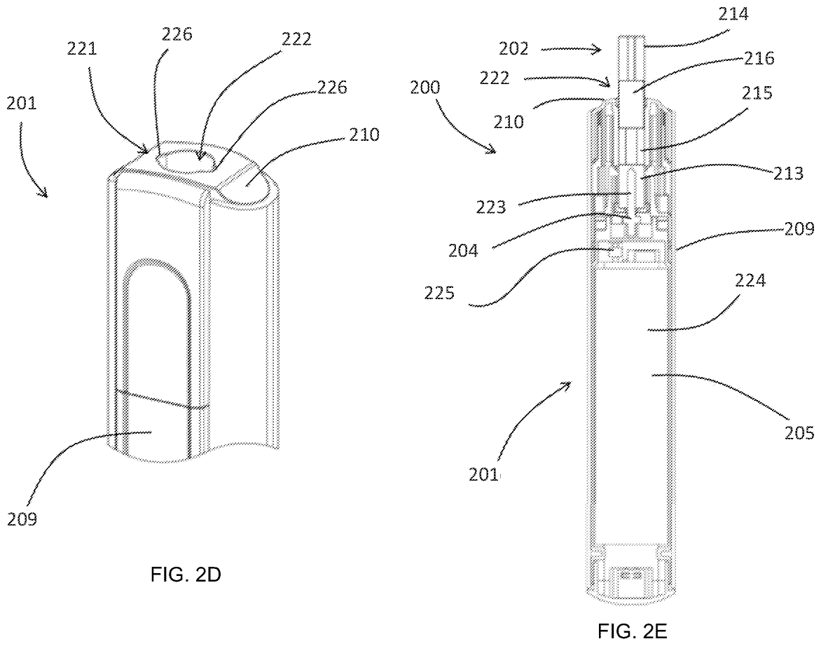

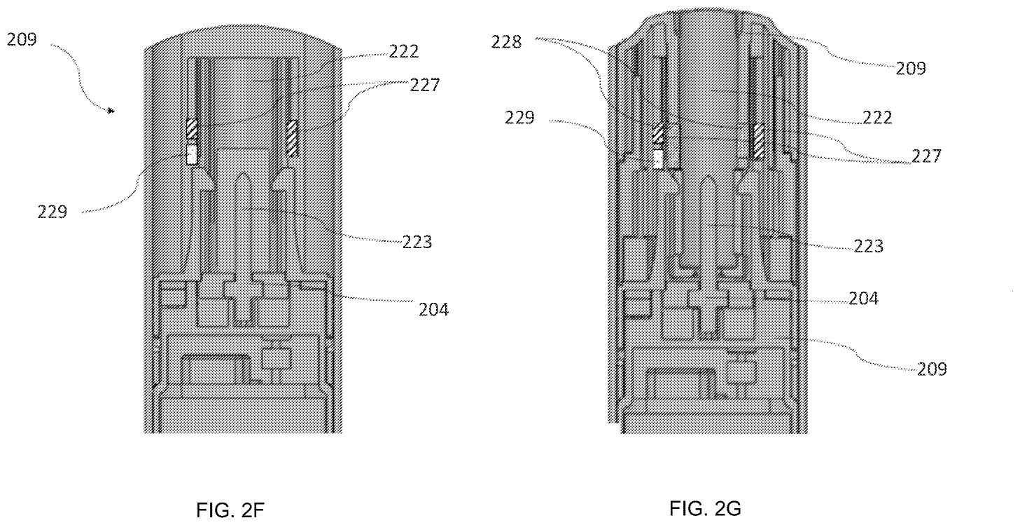

FIG. 2A and FIG. 2B illustrate a heated-tobacco (HT) smoking substitute system 200. The system 200 is an example of the systems 100, 100′ described in relation to FIG. 1A or FIG. 1B. System 200 includes an HT device 201 and an HT consumable 202. The description of FIG. 1A and FIG. 1B above is applicable to the system 200 of FIG. 2A and FIG. 2B, and will thus not be repeated.

The device 201 and the consumable 202 are configured such that the consumable 202 can be engaged with the device 201. FIG. 2A shows the device 201 and the consumable 202 in an engaged state, whilst FIG. 2B shows the device 201 and the consumable 202 in a disengaged state.

The device 201 comprises a main body 209 and cap 210. In use the cap 210 is engaged at an end of the main body 209. Although not apparent from the figures, the cap 210 is moveable relative to the main body 209. In particular, the cap 210 is slidable and can slide along a longitudinal axis of the main body 209.

The device 201 comprises an output means (forming part of the UI of the device 201) in the form of a plurality of light-emitting diodes (LEDs) 211 arranged linearly along the longitudinal axis of the device 201 and on an outer surface of the main body 209 of the device 201. A button 212 is also arranged on an outer surface of the main body 209 of the device 201 and is axially spaced (i.e., along the longitudinal axis) from the plurality of LEDs 211.

FIG. 2C show a detailed section view of the consumable 202 of the system 200. The consumable 202 generally resembles a cigarette. In that respect, the consumable 202 has a generally cylindrical form with a diameter of 7 mm and an axial length of 70 mm. The consumable 202 comprises an aerosol forming substrate 213, a terminal filter element 214, an upstream filter element 215 and a spacer element 216. In other embodiments, the consumable may further comprise a cooling element. A cooling element may exchange heat with vapor that is formed by the aerosol-forming substrate 213 in order to cool the vapor so as to facilitate condensation of the vapor.

The aerosol-forming substrate 213 is substantially cylindrical and is located at an upstream end 217 of the consumable 202, and comprises the aerosol former of the system 200. In that respect, the aerosol forming substrate 213 is configured to be heated by the device 201 to release a vapor. The released vapor is subsequently entrained in an airflow flowing through the aerosol-forming substrate 213. The airflow is produced by the action of the user drawing on a downstream 218 (i.e., terminal or mouth) end of the consumable 202.

In the present embodiment, the aerosol forming substrate 213 comprises tobacco material that may, for example, include any suitable parts of the tobacco plant (e.g., leaves, stems, roots, bark, seeds and flowers). The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon). For example, the aerosol-forming substrate 213 may comprise a gathered sheet of homogenized (e.g., paper/slurry recon) tobacco or gathered shreds/strips formed from such a sheet.

In order to generate an aerosol, the aerosol forming substrate 213 comprises at least one volatile compound that is intended to be vaporized/aerosolized and that may provide the user with a recreational and/or medicinal effect when inhaled. The aerosol-forming substrate 213 may further comprise one or more additives. For example, such additives may be in the form of humectants (e.g., propylene glycol and/or vegetable glycerin), flavorants, fillers, aqueous/non-aqueous solvents and/or binders.

The terminal filter element 214 is also substantially cylindrical, and is located downstream of the aerosol forming substrate 213 at the downstream end 218 of the consumable 202. The terminal filter element 214 is in the form of a hollow bore filter element having a bore 219 (e.g., for airflow) formed therethrough. The diameter of the bore 219 is 2 mm. The terminal filter element 214 is formed of a porous (e.g., monoacetate) filter material. As set forth above, the downstream end 218 of the consumable 202 (i.e., where the terminal filter 214 is located) forms a mouthpiece portion of the consumable 202 upon which the user draws. Airflow is drawn from the upstream end 217, thorough the components of the consumable 202, and out of the downstream end 218. The airflow is driven by the user drawing on the downstream end 218 (i.e., the mouthpiece portion) of the consumable 202.

The upstream filter element 215 is located axially adjacent to the aerosol-forming substrate 213, between the aerosol-forming substrate 213 and the terminal filter element 214. Like the terminal filter 214, the upstream filter element 215 is in the form of a hollow bore filter element, such that it has a bore 220 extending axially therethrough. In this way, the upstream filter 215 may act as an airflow restrictor. The upstream filter element 215 is formed of a porous (e.g., monoacetate) filter material. The bore 220 of the upstream filter element 215 has a larger diameter (3 mm) than the terminal filter element 214.

The spacer 216 is in the form of a cardboard tube, which defines a cavity or chamber between the upstream filter element 215 and the terminal filter element 214. The spacer 216 acts to allow both cooling and mixing of the vapor/aerosol from the aerosol-forming substrate 213. The spacer has an external diameter of 7 mm and an axial length of 14 mm.

Although not apparent from the figure, the aerosol-forming substrate 213, upstream filter 215 and spacer 216 are circumscribed by a paper wrapping layer. The terminal filter 214 is circumscribed by a tipping layer that also circumscribes a portion of the paper wrapping layer (so as to connect the terminal filter 214 to the remaining components of the consumable 202). The upstream filter 215 and terminal filter 214 are circumscribed by further wrapping layers in the form of plug wraps.

Returning now to the device 201, FIG. 2D illustrates a detailed view of the end of the device 201 that is configured to engage with the consumable 202. The cap 210 of the device 201 includes an opening 221 to an internal cavity 222 (more apparent from FIG. 2D) defined by the cap 210. The opening 221 and the cavity 222 are formed so as to receive at least a portion of the consumable 202. During engagement of the consumable 202 with the device 201, a portion of the consumable 202 is received through the opening 221 and into the cavity 222. After engagement (see FIG. 2B), the downstream end 218 of the consumable 202 protrudes from the opening 221 and thus also protrudes from the device 201. The opening 221 includes laterally disposed notches 226. When a consumable 202 is received in the opening 221, these notches 226 remain open and could, for example, be used for retaining a cover in order to cover the end of the device 201.

FIG. 2E shows a cross section through a central longitudinal plane through the device 201. The device 201 is shown with the consumable 202 engaged therewith.

The device 201 comprises a heater 204 comprising heating element 223. The heater 204 forms part of the main body 209 of the device 201 and is rigidly mounted to the main body 209. In the illustrated embodiment, the heater 204 is a rod heater with a heating element 223 having a circular transverse profile. In other embodiments the heater may be in the form of a blade heater (e.g., heating element with a rectangular transverse profile) or a tube heater (e.g., heating element with a tubular form).

The heating element 223 of the heater 204 projects from an internal base of the cavity 222 along a longitudinal axis towards the opening 221. As is apparent from the figure, the length (i.e., along the longitudinal axis) of the heating element is less than a depth of the cavity 222. In this way, the heating element 223 does not protrude from or extend beyond the opening 221.

When the consumable 202 is received in the cavity 222 (as is shown in FIG. 2E), the heating element 223 penetrates the aerosol-forming substrate 213 of the consumable 202. In particular, the heating element 223 extends for nearly the entire axial length of the aerosol-forming substrate 213 when inserted therein. Thus, when the heater 204 is activated, heat is transferred radially from an outer circumferential surface the heating element 223 to the aerosol-forming substrate 213.

The device 201 further comprises an electronics cavity 224. A power source, in the form of a rechargeable battery 205 (a lithium-ion battery), is located in electronics cavity 224.

The device 201 includes a connector (i.e., forming part of an IO module of the device 201) in the form of a USB port. The connector may alternatively be, for example, a micro-USB port or a USB-C port for examples. The USB port may be used to recharge the rechargeable battery 205.

The device 201 includes a controller (not shown) located in the electronics cavity 224. The controller comprises a microcontroller mounted on a printed circuit board (PCB). The USB port is also connected to the controller (i.e., connected to the PCB and microcontroller).

The controller (not shown) is configured to control at least one function of the device 201. For example, the controller is configured to control the operation of the heater 204. Such control of the operation of the heater 204 may be accomplished by the controller toggling the electrical connection of the rechargeable battery 205 to the heater 204. For example, the controller is configured to control the heater 204 in response to a user depressing the button 212. Depressing the button 212 may cause the controller to allow a voltage (from the rechargeable battery 205) to be applied to the heater 204 (so as to cause the heating element 223 to be heated).

The controller is also configured to control the LEDs 211 in response to (e.g., a detected) a condition of the device 201 or the consumable 202. For example, the controller may control the LEDs to indicate whether the device 201 is in an on state or an off state (e.g., one or more of the LEDs may be illuminated by the controller when the device is in an on state).

The device 201 comprises a further input means (i.e., in addition to the button 212) in the form of a puff sensor 225. The puff sensor 225 is configured to detect a user drawing (i.e., inhaling) at the downstream end 218 of the consumable 202. The puff sensor 225 may, for example, be in the form of a pressure sensor, flowmeter or a microphone. The puff sensor 225 is operatively connected to the controller in the electronics cavity 224, such that a signal from the puff sensor 225, indicative of a puff state (i.e., drawing or not drawing), forms an input to the controller (and can thus be responded to by the controller).

The device 201 further comprises means to help retain the cap 210 on the main body 209 of the device 201 when engaged therewith. This is illustrated in FIG. 2F and FIG. 2G, which respectively show the cap 210 in an open position (removed from the main body 209) and a closed position (engaged with the main body 209). As is shown in these figures, the main body 209 comprises magnets 227 mounted in a wall of the main body 209 that defines the cavity 222. In particular, the magnets 227 are mounted such that they each define a portion of the wall of the cavity 222 and so as to oppose each other either side of the heating element 223. As is apparent in particular from FIG. 2G, the cap 210 also comprises two corresponding magnets 228. These magnets 228 are mounted to a portion of the cap 210 that is received in the cavity 222 when the cap 210 is engaged with the main body 209. When the cap 210 is in the closed position, the magnets 228 of the cap 210 align with the magnets 227 of the main body 209 so as to magnetically interact. In this way, the cap 210 is at least partly retained on the main body 209 by the magnets 227, 228. The magnets 227, 228 also assist a user in engaging the cap 210 with the main body 209 (i.e., by magnetic attraction). The magnets 227, 228 may thus provide a form of feedback to the user (i.e., such that a user knows when the cap 210 is correctly engaged). Further, the magnets 227, 228 ensure the cap 210 is aligned on the main body 209. This may, for example, ensure that the cap 210 takes the same position on the main body 209 each time it is engaged with the main body 209. This may help to ensure, for example, that air passages between the cap 210 and the main body 209 are consistent.

The device 201 further comprises a Hall effect sensor 229. As will be described further below, the Hall effect sensor 229 allows the device 201 to detect whether the cap 210 is an open or closed position. In the present case, in the open position, the cap 210 is fully disengaged with the main body 209 of the device 210.

The Hall effect sensor 229 is disposed on the main body 209 at a wall defining the cavity 222. As is apparent from FIG. 2G, when the cap 210 is in the closed position (received in the cavity 222) the Hall effect sensor 229 aligns with a magnet 228 of the cap 210. When the cap 210 is in this position, the Hall effect sensor 229 detects the presence of the magnet 228 and transmits a signal (in the form of a voltage) to the controller of the device 201 (e.g., by wired connection with the controller). When the cap 210 is in the open position, the Hall effect sensor 229 does not detect the presence of the magnet 228 and no signal is transmitted to the controller. Thus, a lack of signal (or voltage) is indicative of the cap 210 being in the open position. In response to a lack of signal from the Hall effect sensor 229, the controller may operate as described above. That is, the controller may prevent activation of the heater 204. For example, the controller may not cause activation of the heater 204 even when signaled to do so by a user input. The prevention of activation of the heater 204 may be performed by a restriction or prevention of power supply from the source 205 to the heater 204.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1.-192. (canceled)

193. A smoking substitute device, comprising:
a main body;
a heater;
a cap engageable with the main body and movable between a closed position in which it substantially encloses the heater and an open position wherein at least a portion of the heater is exposed;
a sensor for detecting a position of the cap; and
a controller configured to control the device in response to the detection of the position of the cap.

194. The smoking substitute device according to claim 193, wherein the controller is configured to prevent activation of the heater when the sensor detects that the cap is in the open position.

195. The smoking substitute device according to claim 194, wherein, in the open position, the cap is disengaged from the main body.

196. The smoking substitute device according to claim 194, comprising a power source for supplying power to the heater, and wherein the controller prevents activation of the heater by preventing supply of power from the power source to the heater.

197. The smoking substitute device according to claim 194, comprising a magnet disposed on the cap or main body, wherein the sensor is disposed on the other of the cap or the main body, the sensor configured to detect the presence of the magnet when the cap is engaged with the main body.

198. The smoking substitute device according to claim 197, wherein the magnet is disposed on the cap and the sensor is disposed on the main body.

199. The smoking substitute device according to claim 198, wherein the body comprises a cavity for receipt of at least a portion of the cap, the sensor mounted to a wall defining the cavity and the magnet mounted to the at least a portion of the cap received in the cavity.

200. The smoking substitute device according to claim 193, wherein the sensor is a Hall effect sensor.

201. The smoking substitute device according to claim 193, comprising a magnet disposed on the cap or main body and a ferromagnetic element disposed on the other of the cap or the main body, the ferromagnetic element and magnet arranged so as to magnetically interact when the cap is in the closed position.

202. The smoking substitute device according to claim 193 that is a heat-not-burn device.

203. A method of controlling a smoking substitute device, the method comprising detecting a state of a cap of the device and controlling a heater of the device in response to the detected state of the cap.

204. The method according to claim 203, wherein the state of the cap comprises whether the cap is in an open position in which the heater is exposed or a closed position in which the heater is substantially enclosed by the cap.

205. The method according to claim 204, wherein controlling the heater comprises preventing activation of the heater when the cap is in the open position.

206. A smoking substitute system, comprising:
the smoking substitute device according to claim 193; and
an aerosol forming article.

207. The smoking substitute system according to claim 206, wherein the aerosol forming article is a heat-not-burn consumable.

208.-295. (canceled)

296. The smoking substitute device according to claim 193, wherein, in response to a detection that the cap is in the open position, the controller is configured to reduce a temperature of the heater, prevent activation of the heater, lock a user input of the device to prevent user interaction with the device, or not respond to a user input.

297. The smoking substitute device according to claim 193, wherein the cap has an opening configured to receive a smoking substitute article for engaging the smoking substitute article with the heater when the cap is in the closed position.

298. The smoking substitute device according to claim 297, wherein the cap is engaged with the main body in both the open position and the closed position.

299. The smoking substitute device according to claim 193, wherein the cap is engaged with the main body in both the open position and the closed position.

300. The smoking substitute device according to claim 193, wherein the heater is exposed laterally when the cap is in the open position.
